(19) United States
(12) Patent Application Publication
(10) Pub. No.: US 2024/0108592 A1
(43) Pub. Date: Apr. 4, 2024

(54) COMBINATION THERAPY WITH IMMUNOTHERAPEUTIC AGENT FOR CANCER

(71) Applicant: Gongwin Biopharm Co., Ltd, Taipei City (TW)

(72) Inventors: Shun-Chi WU, Taipei City (TW); Chuan-Ching YANG, Taipei City (TW); Zong-Yu YANG, Taipei City (TW); Chia-En LIN, Taipei City (TW); Mao-Yuan LIN, Taipei City (TW)

(73) Assignee: Gongwin Biopharm Co., Ltd, Taipei City (TW)

(21) Appl. No.: 18/369,853

(22) Filed: Sep. 19, 2023

Related U.S. Application Data
(60) Provisional application No. 63/376,197, filed on Sep. 19, 2022.

(51) Int. Cl. A61K 31/18; A61K 39/395; A61P 35/00

(52) U.S. Cl. CPC A61K 31/18; A61K 39/39558; A61P 35/00; A61K 2039/545

(57) ABSTRACT

Provided is a method for treating cancer by administering to a subject in need thereof with a pharmaceutical composition including a benzenesulfonamide derivative in combination with a cancer immunotherapeutic agent such as the immune check point inhibitor (ICI).

This application claims the benefit of priority under 35 USC § 119(e) of U.S. Provisional Patent Application No. 63/376,197 filed on Sep. 19, 2022, the contents of which are incorporated by reference as if fully set forth herein in their entirety.

### BACKGROUND

### Technical Field

The present disclosure relates to a method for the treating proliferative diseases, particularly to the method for treating cancer by a combination therapy.

### Description of the Related Art

There were an estimated 18.1 million cancer cases around the world in 2020. By 2040, the global burden is expected to grow to 27.5 million new cancer cases and 16.3 million cancer deaths.

There are approximately more than 700 oncology drugs, and more are still developing. Chemotherapy is one of the common cancer treatments that involves the disruption of cell metabolism and can be more effective than other treatment choices. However, the resistance thereof may occur as the individual's genetic differences, especially in tumor somatic cells. Also, the drug resistance can be occurred through different mechanisms, for example, cell death inhibition and gene mutation.

Other forms of therapies such as radiation, hormone therapy, molecular targeted therapy and surgery are often unresponsive and ineffective.

Since the treatment of cancer is still unsatisfied, there is a clear unmet medical need for the development of combination strategies for all types of cancers.

### SUMMARY

In view of the above-mentioned problems in the technical field, the present disclosure provides effective methods for the treatment of proliferative diseases such as cancer.

Cancer immunotherapy comes in a variety of forms, including targeted antibodies, cancer vaccines, adoptive cell transfer, oncolytic viruses, immune checkpoint inhibitors (ICIs), cytokines, and adjuvants. Immunotherapies are a form of biotherapy (also called biologic therapy or biological response modifier therapy). Some immunotherapy treatments use genetic engineering to enhance cancer-fighting capabilities of the immune cells and may be referred to as gene therapies. Many immunotherapy treatments for preventing, managing, or treating different cancers can also be used in combination with surgery, chemotherapy, radiation, or targeted therapies to improve their effectiveness.

The present disclosure provides a method of treating cancer comprising administering a therapeutically effective amount of a pharmaceutical composition and an immunotherapeutic agent to a subject in need thereof, wherein the pharmaceutical composition comprises a benzenesulfonamide derivative and a pharmaceutically acceptable carrier thereof.

In at least one embodiment of the present disclosure, the benzenesulfonamide derivative is represented by formula (I) below:

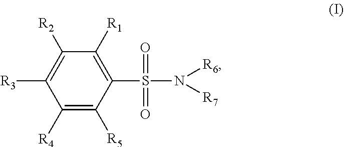

or a pharmaceutically acceptable salt thereof,
wherein R₁ to R₇ are independently selected from the group consisting of H, a C₁-C₆ linear or branched alkyl group, a C₁-C₆ linear or branched alkoxy group, a C₃-C₆ cycloalkyl group, a C₃-C₆ cycloheteroalkyl group, an amino group, and a halo group, or R₆ and R₇ are linked to each other to form a ring, and
wherein the alkyl group, the alkoxy group, the cycloalkyl group, the cycloheteroalkyl group, and the ring in R₁ to R₇ are independently unsubstituted or substituted with one or more substituents.

In at least one embodiment of the present disclosure, the substituent is selected from the group consisting of phenyl, halo, oxo, ether, hydroxyl, carboxyl, amino, sulfo and sulfonamide group.

In at least one embodiment of the present disclosure, the benezesulfonamide derivative or the pharmaceutically acceptable salt thereof is selected from the group consisting of para-toluene sulfonamide, ortho-toluene sulfonamide, meta-toluene sulfonamide, N-ethyl-ortho-toluene sulfonamide, N-ethyl-para-toluene sulfonamide, and N-cyclohexyl-para-toluene sulfonamide. In some embodiments, the benzenesulfonamide derivative is para-toluene sulfonamide.

In some embodiments, the immunotherapeutic agent is a targeted antibody, a cancer vaccine, an adoptive cell transfer, an oncolytic virus, an immune checkpoint inhibitor (ICIs), a cytokine, and an adjuvant. In at least one embodiment of the present disclosure, the immunotherapeutic agent is an ICI.

ICIs are a novel class of immunotherapy drugs that can improve the treatment of a broad spectrum of cancers including, but not limited to metastatic melanoma, non-small lung cancer, or renal cell carcinoma. These humanized monoclonal antibodies target inhibitory receptors (e.g., CTLA-4, PD-1, LAG-3, TIM-3) and ligands (PD-L1) expressed on T lymphocytes, antigen presenting cells and tumor cells and elicit an anti-tumor response by stimulating immune system. In addition, the broad-spectrum antitumor activity and good tolerability of ICIs also make it the backbone of a burgeoning number of clinical trials testing combination regimens with other immunomodulatory agents or conventional systemic anticancer therapy including, but not limited to, cytotoxic chemotherapy or molecular targeted therapy.

In at least one embodiment, the ICI comprises anti-PD-1 antibodies, anti-PD-L1 antibodies, anti-CTLA4 antibodies, anti-LAG-3 antibodies, anti-TIM-3 antibodies, or any combinations thereof.

In some embodiments, the cancer is melanoma, or carcinoma of the head and neck, brain, nervous system, thyroid, thymus, esophagus, stomach, lung, breast, gastrointestinal tract, colorectum, liver, pancreas, kidney, adrenal cortex, genitourinary system, prostate, bladder, urothelium, uterus, cervix, ovary, skin, or hematologic malignancy. In at least one embodiment, the cancer is small cell lung cancer (SCLC), non-small cell lung cancer (NSCLC), squamous carcinoma of lung or adenocarcinoma of lung. In other embodiments, the cancer is primary cancer or secondary cancer.

In some embodiments, the cancer is a carcinoma in situ, a localized cancer, a regional cancer, or an advanced cancer. In some embodiments, the cancer is a metastatic cancer.

In some embodiments, the cancer is an early stage cancer, a late stage cancer, a low-grade cancer, or a high grade cancer.

In at least one embodiment, the cancer is a solid tumor or a non-solid tumor. In other embodiments, the cancer is a sarcoma, a carcinoma, a lymphoma, or a leukemia.

In at least one embodiment of the present disclosure, the cancer is a melanoma or lung cancer. In at least one embodiment of the present disclosure, the cancer is a breast cancer or a melanoma.

In some embodiments, a ratio of the benzenesulfonamide derivative to the immunotherapeutic agent is in a range of 12.5:1 to 2000:1. In other embodiments, the benzenesulfonamide derivative is administered to the subject at a dosage of about 55 mg/kg, and the immunotherapeutic agent is administered to the subject at a dosage of about 2 mg/kg. In some embodiments, a ratio of the benzenesulfonamide derivative to the immunotherapeutic agent is in a range of 1.5:1 to 100:1. In other embodiments, the benzenesulfonamide derivative is administered to the subject at a dosage of about 330 mg/kg, and the immunotherapeutic agent is administered to the subject at a dosage of about 10 mg/kg.

In some embodiments, a ratio of the benzenesulfonamide derivative to the immunotherapeutic agent is in a range of 1.5:1 to 100:1. In other embodiments, the benzenesulfonamide derivative is administered to the subject at a dosage of about 165 mg/kg, and the immunotherapeutic agent is administered to the subject at a dosage of about 2 mg/kg.

In at least one embodiment of the present disclosure, the benzenesulfonamide derivative in the pharmaceutical composition is administered to the subject in an effective amount of from about 3,300 mg to about 26,400 mg.

In at least one embodiment of the present disclosure, the benzenesulfonamide derivative in the pharmaceutical composition is administered to the subject in an effective amount of from about 165 mg to about 6,600 mg per day.

In at least one embodiment of the present disclosure, the pharmaceutical composition or the immunotherapeutic agent is administered to the subject one time to five times a week. In other embodiments, the pharmaceutical composition is administered to the subject two times a week, and the immunotherapeutic agent is administered to the subject one time a week. In at least one embodiment of the present disclosure, the pharmaceutical composition or the immunotherapeutic agent is administered to the subject one time a week.

In at least one embodiment of the present disclosure, the pharmaceutically acceptable carrier is selected from the group consisting of a filler, a binder, a preservative, a disintegrating agent, a lubricant, a suspending agent, a wetting agent, a flavoring agent, a thickening agent, an acid, a biocompatible solvent, a surfactant, a complexation agent, and any combination thereof.

In at least one embodiment of the present disclosure, the pharmaceutically acceptable carrier is selected from the group consisting of polyethylene glycol, alkylene glycol, propylene glycol, sebacic acid, dimethyl sulfoxide, ethanol, and any combination thereof.

In at least one embodiment of the present disclosure, the pharmaceutical composition is in a form selected from the group consisting of a formulation to injection, dry powder, a tablet, an oral liquid, a wafer, a film, a lozenge, a capsule, a granule, a pill, a gel, a lotion, an ointment, an emulsifier, a paste, a cream, an eye drop, and a salve.

In at least one embodiment of the present disclosure, the pharmaceutical composition or the anti-cancer agent is administered to the subject intratumorally, intravenously, subcutaneously, intradermally, orally, intrathecally, intraperitoneally, intranasally, intramuscularly, intrapleuraly, topically, or through nebulization.

In at least one embodiment of the present disclosure, the subject is a human, a dog, a cat, or a mouse.

According to the present disclosure, the immunotherapeutic agent can be a therapeutic antibody, including, but not limiting to, Herceptin (Trastuzumab) (Genentech, California), which is a humanized anti-HER2 monoclonal antibody for the treatment of patients with metastatic breast cancer; Reopro (abciximab) (Centocor), which is an anti-glycoprotein receptor on the platelets for the prevention of clot formation; Zenapax (daclizumab) (Roche Pharmaceuticals, Switzerland), which is an immunosuppressive, humanized anti-CD25 monoclonal antibody for the prevention of acute renal allograft rejection; Panorex, which is a murine anti-17-IA cell surface antigen IgG2a antibody (Glaxo Wellcome/Centocor); BEC2, which is a murine anti-idiotype (GD3 epitope) IgG antibody (ImClone System); IMC-C225, which is a chimeric anti-EGFR IgG antibody (ImClone System); Vitaxin, which is a humanized anti-alpha.V.beta.3 integrin antibody (Applied Molecular Evolution/MedImmune); Campath 1H/LDP-03, which is a humanized anti-CD52 IgG1 antibody (Leukosite); Smart M195, which is a humanized anti-CD33 IgG antibody (Protein Design Lab/Kanebo); Rituxan, which is a chimeric anti-CD20 IgG1 antibody (IDEC Pharm/Genentech, Roche/Zettyaku); Lymphocide, which is a humanized anti-CD22 IgG antibody (Immunomedics); Lymphocide Y-90 (Immunomedics); Lymphoscan (Tc-99m-labeled; radioimaging; Immunomedics); Nuvion (against CD3; Protein Design Labs); CM3, which is a humanized anti-ICAM3 antibody (ICOS Pharm); IDEC-114, which is a primatied anti-CD80 antibody (IDEC Pharm/Mitsubishi); Zevalin, which is a radiolabelled murine anti-CD20 antibody (IDEC/Schering AG); IDEC-131, which is a humanized anti-CD40L antibody (IDEC/Eisai); IDEC-151, which is a primatized anti-CD4 antibody (IDEC); IDEC-152, which is a primatized anti-CD23 antibody (IDEC/Seikagaku); SMART anti-CD3, which is a humanized anti-CD3 IgG (Protein Design Lab); 5G1.1, which is a humanized anti-complement factor 5 (C5) antibody (Alexion Pharm); D2E7, which is a humanized anti-TNF-alpha antibody (CAT/BASF); CDP870, which is a humanized anti-TNF-alpha Fab fragment (Celltech); IDEC-151, which is a primatized anti-CD4 IgG1 antibody (IDEC Pharm/SmithKline Beecham); MDX-CD4, which is a human anti-CD4 IgG antibody (Medarex(Eisai/Genmab)); CD20-sreptdavidin (+biotin-yttrium 90; NeoRx); CDP571, which is a humanized anti-TNF-alpha IgG4 antibody (Celltech); LDP-02, which is a humanized anti-alpha-4-beta-7 antibody (LeukoSite/Genentech); OrthoClone OKT4A, which is a humanized anti-CD4 IgG antibody (Ortho Biotech); Antova, which is a humanized anti-CD40L IgG antibody (Biogen); Antegren, which is a humanized anti-VLA-4 IgG antibody (Elan); and CAT-152, which is a human anti-TGF-beta 2 antibody (Cambridge Ab Tech).

In some embodiments, the ICI is an anti-PD-1 antibody, an anti-PD-L1 antibody, an anti-CTLA4 antibody, an anti-LAG-3 antibody, or an anti-TIM-3 antibody. In some embodiments, the anti-PD-1 antibody, the anti-PD-L1 antibody, the anti-CTLA4 antibody, the anti-LAG-3 antibody, or the anti-TIM-3 antibody is Tecentriq (atezolizumab), Imfinzi (durvalumab), Bavencio (avelumab), Yervoy (ipilimumab), Keytruda (pembrolizumab), Opdivo (nivolumab) or Opdualag (nivolumab and relatlimab-rmbw).

The present disclosure also provides a use of a pharmaceutical composition in the manufacture of a medicament for treating cancer, and the pharmaceutical composition comprises the benzenesulfonamide derivative and the pharmaceutically acceptable carrier thereof and is administered to the subject in need thereof with an immunotherapeutic agent as mentioned above. The present disclosure further provides the pharmaceutical composition for use in the treatment of cancer, and the pharmaceutical composition comprises the benzenesulfonamide derivative and the pharmaceutically acceptable carrier thereof and is administered to the subject in need thereof with an immunotherapeutic agent as mentioned above as mentioned above.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present disclosure will become more readily appreciated by reference to the following descriptions in conjunction with the accompanying drawings.

FIGS. 1A-1B show the tumor growth curve during the first 10 days after breast cancer cell line 4T1 inoculation. FIG. 1A shows the average tumor size of all of the fifty mice on Day10. FIG. 1B shows the average tumor size after excluding ten mice with too small tumors. Data are presented as mean±SEM.

FIG. 2 shows the survival curve of 4T1 breast cancer allograft mouse model in each group before sacrifice on Day 30.

FIG. 3 shows the body weight curve of 4T1 breast cancer allograft mouse model in each group from Day 10 to Day 30. Data are presents as mean±SEM.

FIG. 4 shows the tumor growth curve of 4T1 breast cancer allograft mouse model in each group from Day 10 to Day 30 and the tumor growth inhibition index calculated on Day 30. Data are presented as mean±SEM. *p<0.05, *p<0.01, ***p<0.001, ****p<0.0001.

FIG. 5 shows the tumor weight of 4T1 breast cancer allograft mouse model in each group and the tumor weight inhibition index calculated on Day 30. Data are presented as mean±SD. *p<0.05, **p<0.01, ***p<0.001, ****p<0.0001.

FIG. 6 shows the Combination Index (CI) of PTS combined with αPD-1 of 4T1 breast cancer allograft mouse model, which was calculated from the CI equation algorithms using CompuSyn software. CI=1, <1 and >1 indicates additive effect, synergism and antagonism, respectively.

FIGS. 7A-7B show the number of lung metastatic nodules in each group of 4T1 breast cancer allograft mouse model. FIG. 7A shows the statistical results of lung metastatic nodules in all of the mice in the PBS, PTS100, αPD-1 and PTS100+αPD-1 groups. FIG. 7B shows the statistical results of lung metastatic nodules after excluding outliers from each group. Data are presented as mean±SD. *p<0.05, **p<0.01, ***p<0.001, ****p<0.0001.

FIGS. 8A-8B show the correlation analysis between number of lung metastasis and tumor size (mm³) of 4T1 breast cancer allograft mouse model. FIG. 8A shows the analysis result of the correlation between lung metastasis and tumor volume in each group. FIG. 8B shows the analysis result of the correlation between lung metastasis and tumor volume, in which all groups were combined for analysis.

FIGS. 9A-9B show the correlation analysis between number of lung metastasis and tumor weight (g) of 4T1 breast cancer allograft mouse model. FIG. 9A shows the analysis result of the correlation between lung metastasis and tumor weight in each group. FIG. 9B shows the analysis result of the correlation between lung metastasis and tumor weight, in which all groups were combined for analysis.

FIG. 10 demonstrates the tumor appearance in each treatment group of 4T1 breast cancer allograft mouse model.

FIG. 11 demonstrates the lung appearance in each treatment group of 4T1 breast cancer allograft mouse model.

FIGS. 12A-12B show the tumor growth curve during the first 8 days after melanoma cell line B16-F10 inoculation. FIG. 12A shows the average tumor size of all of the fifty mice on Day 8. FIG. 12B shows the average tumor size after excluding ten mice with too small or too big tumors. Data are presented as mean±SEM.

FIG. 13 shows the survival curve of B16-F10 melanoma allograft mouse model in each group before sacrifice on Day 32. *p<0.05, **p<0.01.

FIG. 14 shows the body weight curve of B16-F10 melanoma allograft mouse model in each group from Day 8 to Day 24. Data are mean±SEM.

FIG. 15 shows the tumor growth curve of B16-F10 melanoma allograft mouse model in each group from Day 8 to Day 24 and the tumor growth inhibition index calculated on Day 24. Data are presented as mean±SEM. *p<0.05, **p<0.01, ***p<0.001.

FIG. 16 shows the tumor weight of B16-F10 melanoma allograft mouse model on Day 32. Data are presented as mean±SD. *p<0.05.

FIG. 17 shows the Combination Index (CI) of PTS combined with αPD-1 of B16-F10 melanoma allograft mouse model, which was calculated from the CI equation algorithms using CompuSyn software. CI=1, <1 and >1 indicates additive effect, synergism and antagonism, respectively.

FIG. 18 demonstrates the tumor appearance in each treatment group of B16-F10 melanoma allograft mouse model.

FIG. 19 demonstrates lung appearance in each treatment group of B16-F10 melanoma allograft mouse model.

### DETAILED DESCRIPTIONS OF THE EMBODIMENTS

The following embodiments are provided to illustrate the present disclosure in detail. A person having ordinary skill in the art can easily understand the advantages and effects of the present disclosure after reading the disclosure of this specification, and also can implement or apply in other different embodiments. Therefore, it is possible to modify and/or alter the following embodiments for carrying out this disclosure without contravening its scope for different aspects and applications, and any element or method within the scope of the present disclosure disclosed herein can combine with any other element or method disclosed in any embodiments of the present disclosure.

As used herein, the singular forms “a,” “an,” and “the” include plural referents unless expressly and unequivocally limited to one referent. The term “or” is used interchangeably with the term “and/or” unless the context clearly indicates otherwise.

As used herein, the term “melanoma” refers to any malignant form of cancer that arises from melanocytes. For example, the melanoma may include, but is not limited to, mucosal melanoma, acral melanoma, ocular melanoma, cutaneous melanoma, superficial spreading melanoma, nodular melanoma, polypoid melanoma, lentigo maligna melanoma, desmoplastic melanoma, soft-tissue melanoma, amelanotic melanoma, juvenile melanoma, Harding-Passey melanoma, subungual melanoma, spitzoid melanoma, blue nevus-like melanoma, or any combination thereof. The melanoma may also include, but is not limited to, metastatic melanoma. Ultraviolet (UV) radiation status, epidemiology, histopathological features, genetics, prognosis, and outcomes may be different between these subtypes of melanomas.

The numeral ranges used herein are inclusive and combinable, any numeral value that falls within the numeral scope herein could be taken as a maximum or minimum value to derive the sub-ranges therefrom. For example, it should be understood that the numeral range “1-60%” comprises any sub-ranges between the minimum value of 1% to the maximum value of 60%, such as the sub-ranges from 1% to 50%, from 10% to 60%, and from 20.5% to 40.5%.

The term “about” as used herein when referring to the numerical value is meant to encompass variations of ±20%, ±10%, ±5%, ±1%, ±0.5%, or ±0.1% from the numerical value. Such variations in the numerical value may occur by, e.g., the experimental error, the typical error in measuring or handling procedure for making compounds, compositions, concentrates, or formulations, the differences in the source, manufacture, or purity of starting materials or ingredients used in the present disclosure, or like considerations.

As used herein, “subject” may encompass any vertebrate including, but not limited to, mammals, reptiles, amphibians, and/or fish. However, advantageously, the subject is a mammal such as a human, or an animal mammal such as a domesticated mammal, e.g., a dog, a cat, a horse, or the like, or a production mammal, e.g., a cow, a sheep, a pig, or the like.

As used herein, the terms “comprise,” “comprising,” “include,” “including,” “have,” “having,” “contain,” “containing,” and any other variations thereof are intended to cover a non-exclusive inclusion. For example, when describing an object “comprises” a limitation, unless otherwise specified, it may additionally include other ingredients, elements, components, structures, regions, parts, devices, systems, steps, or connections, etc., and should not exclude other limitations.

PTS is a carbonic anhydrase inhibitor, which has strong inhibitory effect on CA2, 7, 9, and 12. The main function of CA is to maintain intracellular and extracellular acid-base balance. A hypoxic environment is formed in the overgrown tumor tissue, which induces an increase in the expression of CA9/12. This makes cancer cells resistant to the hypoxic environment, and acidifies the tumor microenvironment. The activity of immune cells in the acidic tumor microenvironment is greatly reduced, resulting in immunosuppression and causing neoplastic cells to escape immune surveillance. Theoretically, if PTS can effectively inhibit CA9/12, it will make cancer cells unable to resist the hypoxic environment, thereby reduce the acidification of the tumor microenvironment and enhance the activity of immune cells such microenvironment, and ultimately enhance the therapeutic effect of immunotherapeutic agents such as ICIs.

Without further elaboration, it is believed that one skilled in the art can utilize the present disclosure to its fullest extent on the basis of the preceding description. The following examples are, therefore, to be construed as merely illustrative and not a limitation of the scope of the present disclosure in any way.

### EXAMPLE

Exemplary embodiments of the present disclosure are further described in the following examples, which should not be construed to limit the scope of the present disclosure.

### Preparation Example 1

Pharmaceutical composition of benzenesulfonamides (PTS):

| | |
|---|---|
| p-Toluenesulfonamide | 1%-60% |
| PEG-400 | 10%-40% |
| 1,2-Propylene glycol | 5%-10% |
| Sebacic acid | 1%-5% |
| p-Toluenesulfonic acid | 0%-15% |
| 2-Ethyl-1,3-hexanediol | 10%-20% |
| Dimethyl sulfoxide | 0-10% |
| Ethanol | 0-20% |

p-Toluenesulfonamide is a small organic compound used as a local therapeutic drug that is injected directly into the tumors and has been shown to induce cancer cell death by activating apoptosis and necrosis in hepatocellular carcinoma, non-small-cell lung cancer, and tongue squamous cell carcinoma. The present disclosure also provides the use of the PTS composition as a medicament for treating cancer.

Preparation of the PTS composition of the present disclosure includes the process of: adding and mixing the solvents and adjuvants in a given ratio; heating the mixture to 80° C. to 110° C. with stirring to form a clear oily liquid; gradually adding the sulfa drug with stirring until completely dissolved; filtering and cooling the mixture to obtain the composition of the present disclosure in an oily liquid form.

The preparation of the PTS composition injection may be conducted by some techniques known in the art, e.g., adding an adjuvant and/or solvent to adjust the mixture to an isotonic state, or filtering the mixture by using a microporous filter.

### Example 1: Combined Treatment of PTS and Anti-PD-1 Antibody in Mice Breast Cancer Cell Lines

Materials and Methods

Tumor cell line used in the experiment was mouse breast cancer cell line 4T1. The mouse breast cancer cell line 4T1 was purchased from ATCC. The cells were cultured in a humidified atmosphere (95% air, 5% CO₂) at 37° C. in a medium containing RPMI (Gibco, 31800022) w/1.5 g sodium bicarbonate (Sigma, S5761), 1% HEPES (Gibco, 15630106), 1% P/S (Gibco, 15140-122), 1% sodium pyruvate (Gibco, 25-000-CIS), and 10% FBS (Hyclone, SH30071.03). The cells were routinely passaged by removing the culture medium and detached the cell monolayer with 0.25% trypsin-EDTA (Gibco, 25200056). Add fresh culture medium, aspirate and dispense into new culture flasks.

Reagents

The PTS100 was provided in the same procedure as illustrated in Preparation Example 1. The anti-PD-1 antibody was InVivoMAb anti-mouse PD-1 (Bio X Cell, BE0146). On the day of administration, it was diluted five-fold with PBS (the concentration of the stock solution was 10 mg/mL, diluted to 2 mg/mL). The diluted anti-mouse PD-1 was filled into the 1 ml insulin syringe, removed the bubbles, and adjust final volume according to the test conditions required for the animal experiments.

Animals Study

3×10⁵ 4T1 cell in 100 uL PBS were inoculated subcutaneously into the right flank side of BALB/c female mice (six-week-old; BioLASCO). Tumor growth was tracked by digital caliper every day and treatments were initiated once an average tumor volume for the cohort reached 70 to 100 mm³ using the modified ellipsoid formula (1×w²/2). Mice were then randomly distributed among groups in a manner to maintain equal size distributions across each treatment group. Treatment with PTS100 (495 mg/kg) or saline control was initiated on day 10, and treatments were administered twice per week until study completion by intratumoral injection. Anti-mouse PD-1 (10 mg/kg) or saline control were administered intraperitoneally (i.p.) on days 10, 16, 20, 27.

Statistical Analysis

All data were expressed as mean±standard deviation (SD) or mean±standard error of the mean (SEM). T-tests was used to access for significant differences between pairs of groups. When comparing more than two groups, an analysis of variance (ANOVA) with a post hoc Bonferroni correction was performed. P-values below 0.05, 0.01, and 0.001 were considered significant, very significant, and extremely significant, respectively.

Results

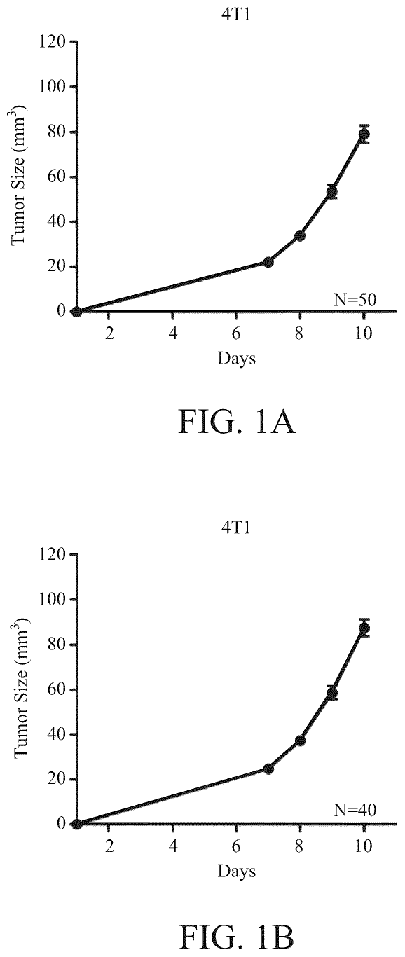

The mouse breast cancer cell line 4T1 was inoculated with 3×10⁵ cells at the right flank side of BALB/c mice, and the average tumor volume of fifty mice on Day 10 was 79.09 mm³ (FIG. 1A). After excluding ten mice with too small tumors, the average tumor volume of the forty mice was 87.49 mm³ (FIG. 1B).

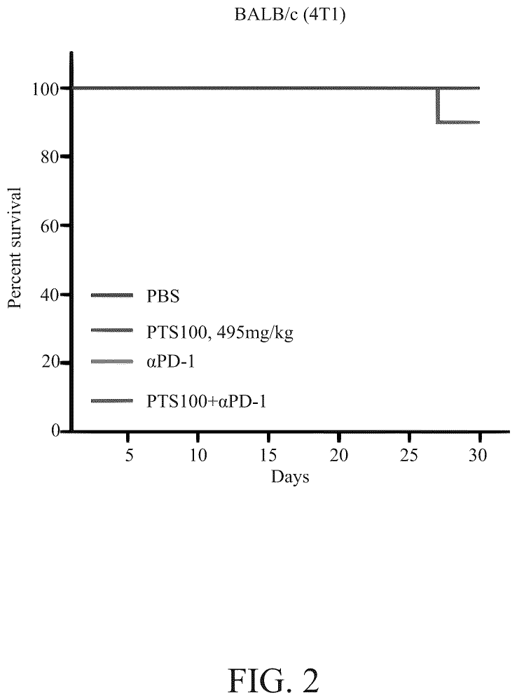

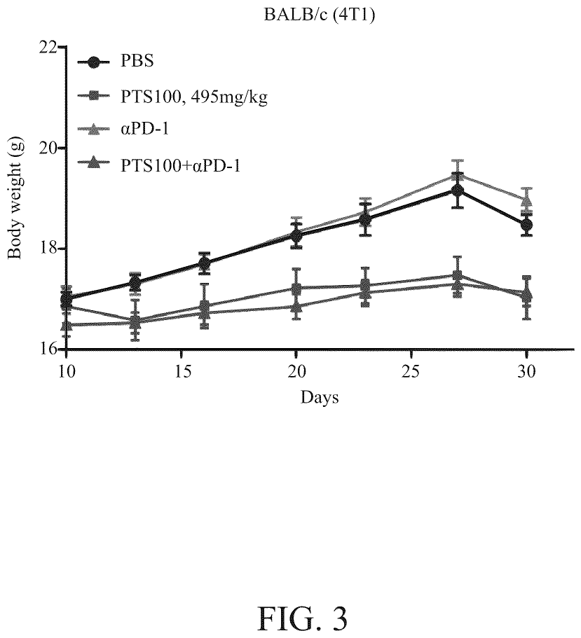

Drug administration started on Day10. Mice were sacrificed on Day30. On Day 27, one mouse in the PTS100+αPD-1 group died unexpectedly due to inadvertent gas anesthesia operation. No other mice died before sacrifice on Day 30 (FIG. 2). Body weight of mice in all groups increased slowly before Day27, but decreased after Day 27 (FIG. 3), which may be caused by the tumor metastasis to the lungs.

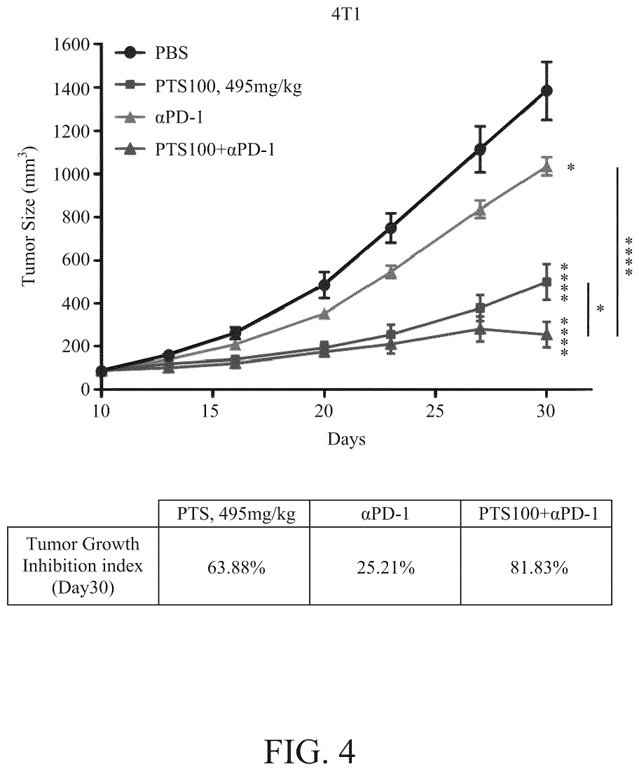

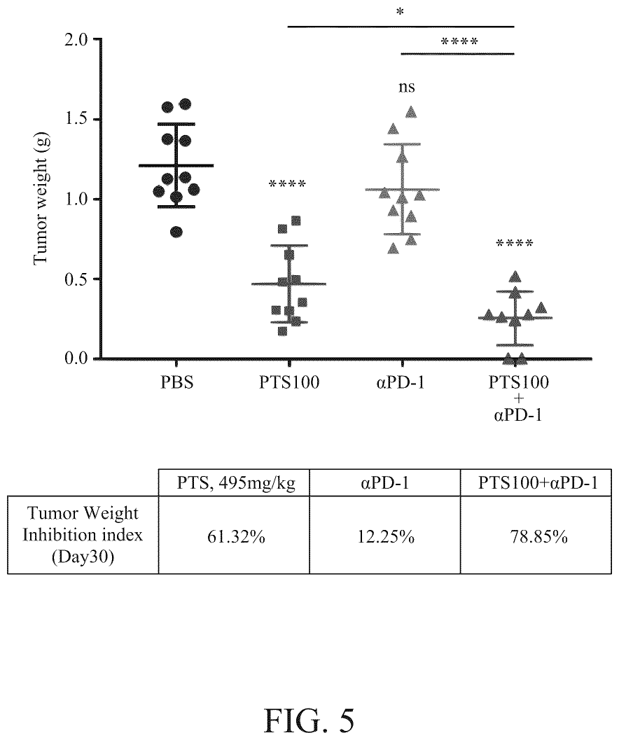

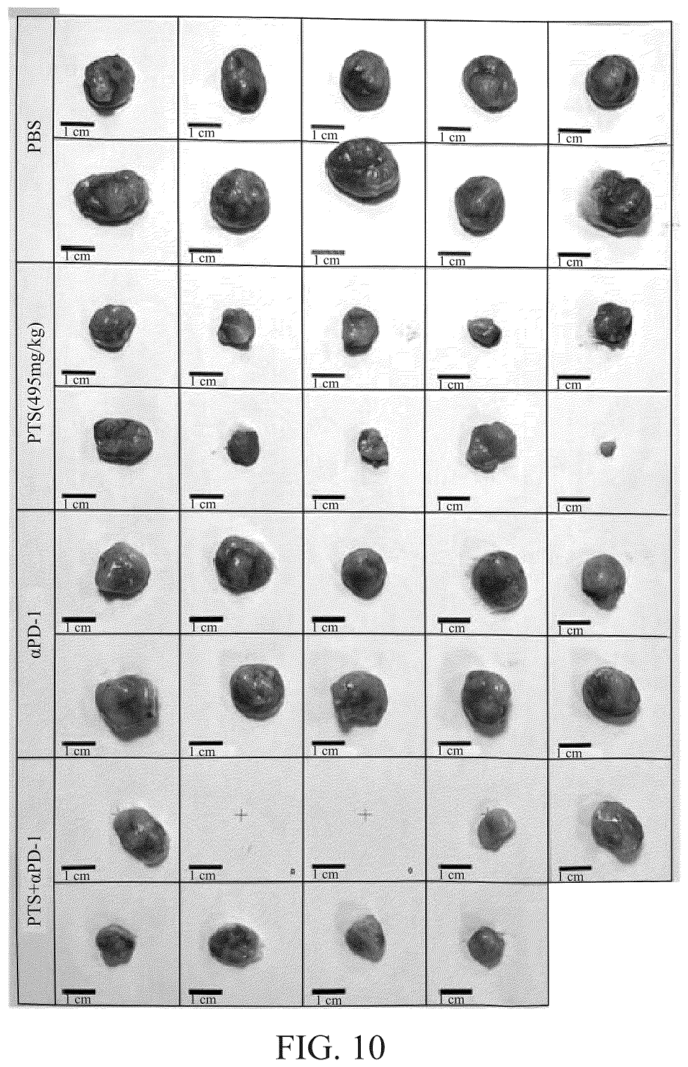

After the 4T1 tumors were treated with PTS100, αPD-1, and PTS100+αPD-1, the tumor growth was significantly reduced (FIG. 4), and the tumor weight in PTS100 and PTS100+αPD-1 groups also significantly reduced (FIG. 5). Two mice in the PTS100+αPD-1 group was complete response to treatment and no tumor tissue was observed (FIG. 10). The tumor growth inhibition rates of the PTS100, αPD-1, and PTS100+αPD-1 groups were 63.88%, 25.21%, and 81.83%, respectively (FIG. 4); and the tumor weight inhibition rates were 61.32%, 12.25%, and 78.85%, respectively (FIG. 5).

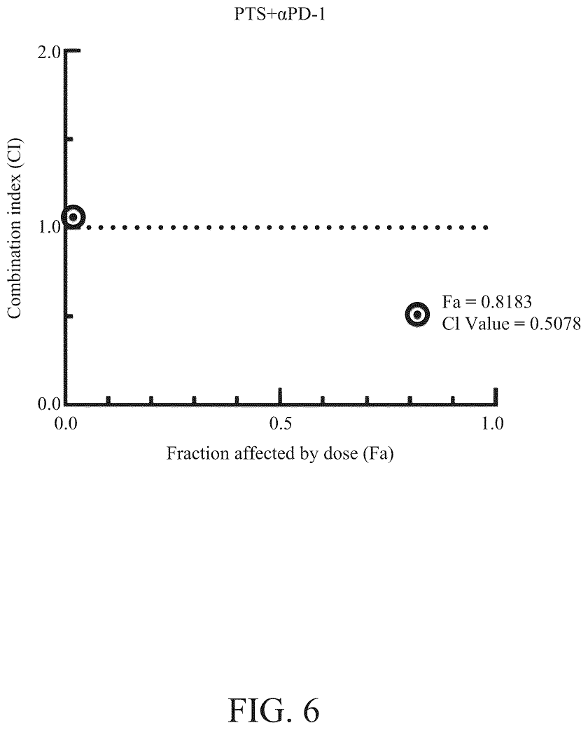

Compared with PTS100 or αPD-1 alone, there was a significant better result of the combination of PTS100+αPD-1 in terms of tumor growth and tumor weight inhibition rates (FIGS. 4 and 5). Through the calculation and analysis using the CompuSyn software, calculating the index of the synergistic effect of drug combination (Combination index, CI, CI=1, <1 and >1 indicates additive effect, synergism and antagonism, respectively), the CI value was 0.5078, confirming the synergistic effect of the drug (FIG. 6).

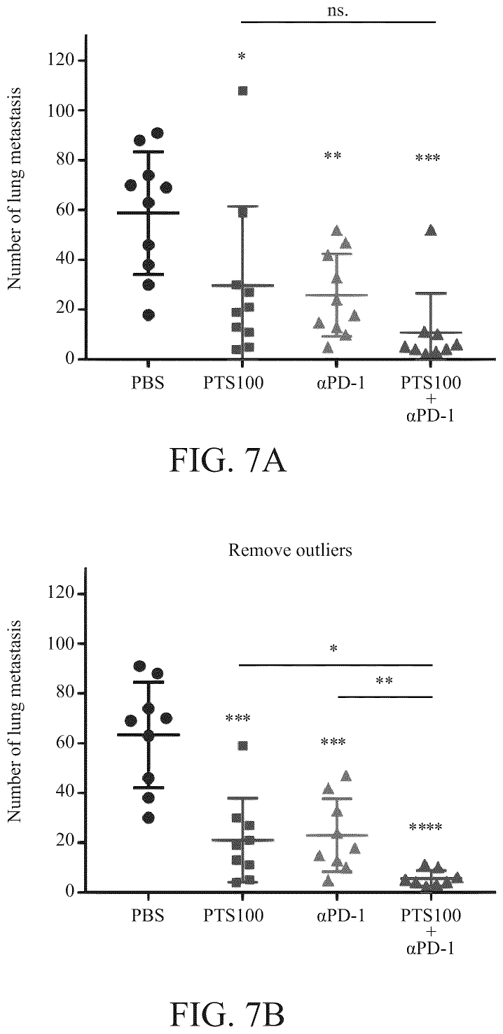

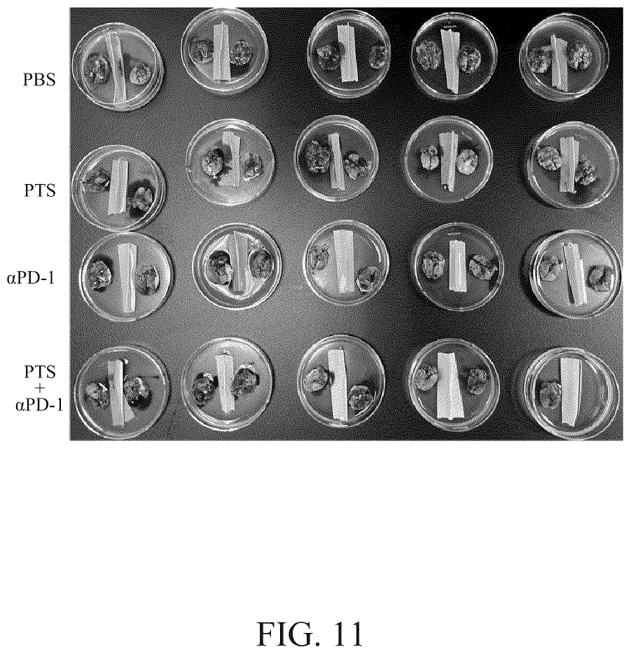

The number of lung metastatic nodules was counted on the day of sacrifice (FIG. 11), soaking them in 10% formalin for at least one day to fix the tissue, then separating all lung lobes and observing and counting the number of nodules with the naked eye. The statistical results showed the number of lung metastatic nodules in the PTS100, αPD-1, and PTS100+αPD-1 groups were significantly lower than those in the PBS group, and all treatment groups had a tendency to have a significantly reduced lung metastases, but had no significant differences between each other (FIG. 7A). Due to the large numerical error in the number of lung metastatic nodules, outliers were excluded from each group for statistics, and the subsequent results showed the group of PTS100+αPD-1 combination treatment had a significantly more decrease in the number of lung metastatic nodules (FIG. 7B) than PTS100 or αPD-1 alone.

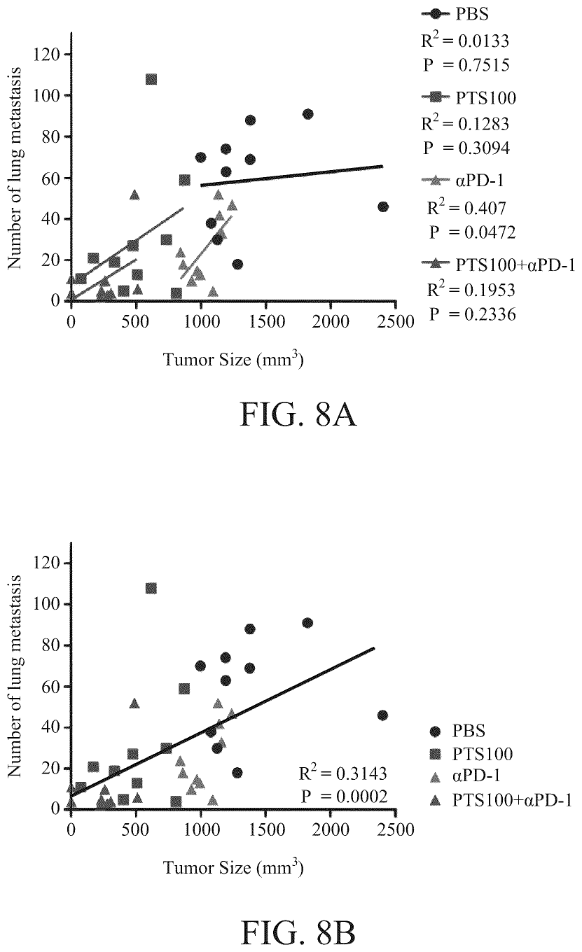

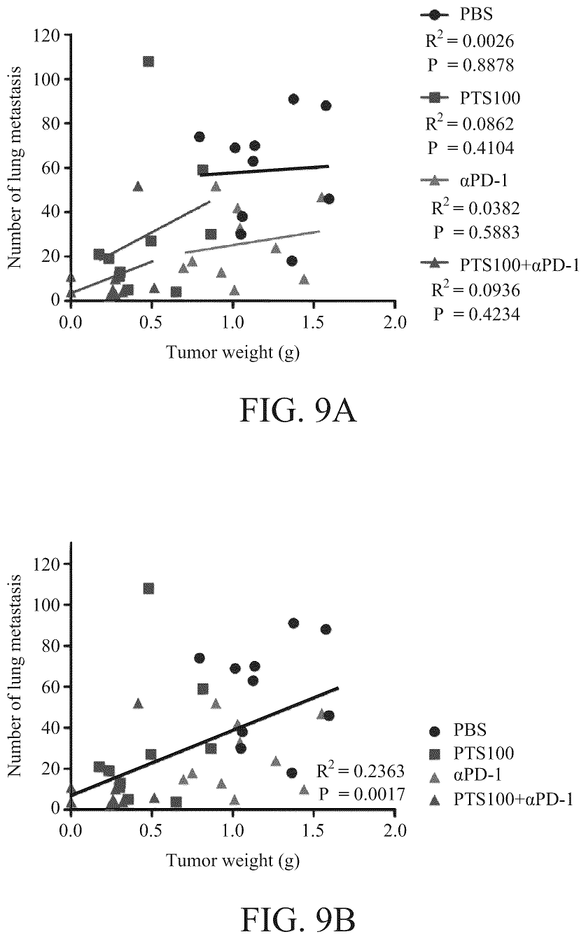

Correlation analysis was carried out between the number of lung metastases nodules and tumor volume (FIGS. 8A-8B) or tumor weight (FIGS. 9A-9B). The results of separate statistics for each group showed a positive correlation (R2>0), but only the αPD-1 group had statistical differences in the analysis of the correlation between lung metastasis and tumor volume (P<0.05) (FIG. 8A). If all groups were combined for correlation analysis (FIG. 8B and FIG. 9B), it could be observed that lung metastasis was significantly positively correlated with tumor volume or tumor weight. This data suggests that the number of lung metastatic nodules is significantly positively correlated with the size of the tumor.

### Example 2: Combined Treatment of PTS and Anti-PD-1 Antibody in Mice Melanoma Cell Lines

Materials and Methods

Tumor Cell Line

Tumor cell line used in the experiment was mouse melanoma cell line B16-F10. The mouse melanoma cell line B16-F10 was purchased from BCRC. The cells were cultured in a humidified atmosphere (95% air, 5% CO₂) at 37° C. in a medium containing DMEM (Gibco, 12800017), w/3.7 g sodium bicarbonate (Sigma, S5761), 1% HEPES (Gibco, 15630106), 1% P/S (Gibco, 15140-122), 1% sodium pyruvate (Gibco, 25-000-CIS), and 10% FBS (Hyclone, SH30071.03). The cells were routinely passaged by removing the culture medium and detached the cell monolayer with 0.25% trypsin-EDTA (Gibco, 25200056). Add fresh culture medium, aspirate and dispense into new culture flasks.

Reagents

The PTS100 and the anti-mouse PD-1 (Bio X Cell, BE0146) that provided in the same procedure as illustrated in Example 1.

Animals Study

5×10⁵ B16-F10 cells in 100 uL PBS were inoculated subcutaneously into the right flank side of C57BL/6 female mice (six-week-old; BioLASCO). Tumor growth was tracked by digital caliper every day and treatments were initiated once an average tumor volume for the cohort reached 60 to 100 mm³ using the modified ellipsoid formula (1×w²/2). Mice were then randomly distributed among groups in a manner to maintain equal size distributions across each treatment group. Treatment with PTS100 (165 mg/kg) or saline control was initiated on day 8, and treatments were administered twice per week until study completion by intratumoral injection. Anti-mouse PD-1 (10 mg/kg) or saline control were administered intraperitoneally (i.p.) on days 8, 14, 21.

Statistical Analysis

All data were expressed and calculated in the same manner as illustrated in Example 1.

Results

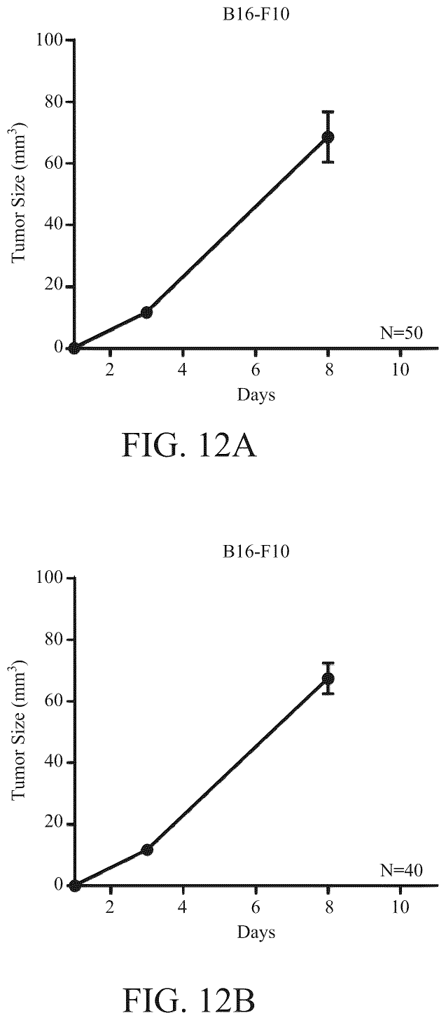

The mouse melanoma cell line B16-F10 was inoculated with 5×10⁵ cells at the right flank side of C57BL/6 mice, and the average tumor volume of fifty mice on Day 8 was 68.63 mm³ (FIG. 12A). After excluding ten mice with too small or too big tumors, the average tumor volume of the forty mice was 67.5 mm³ (FIG. 12B).

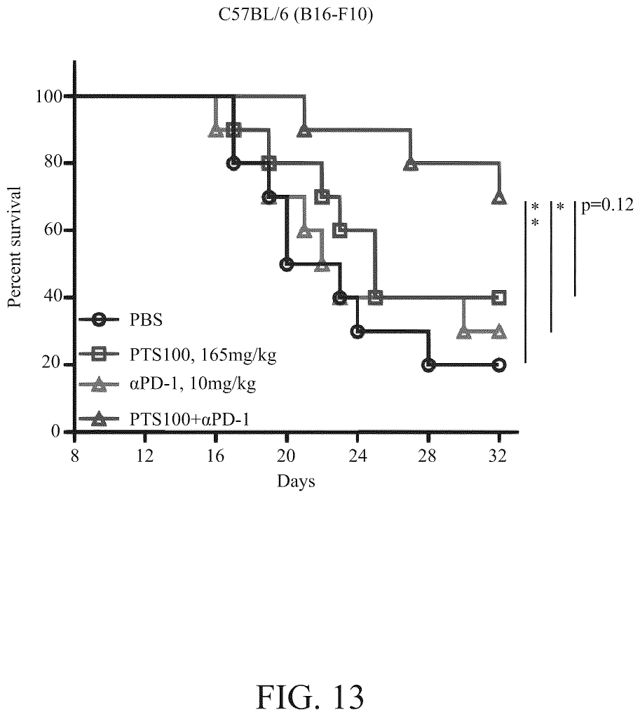

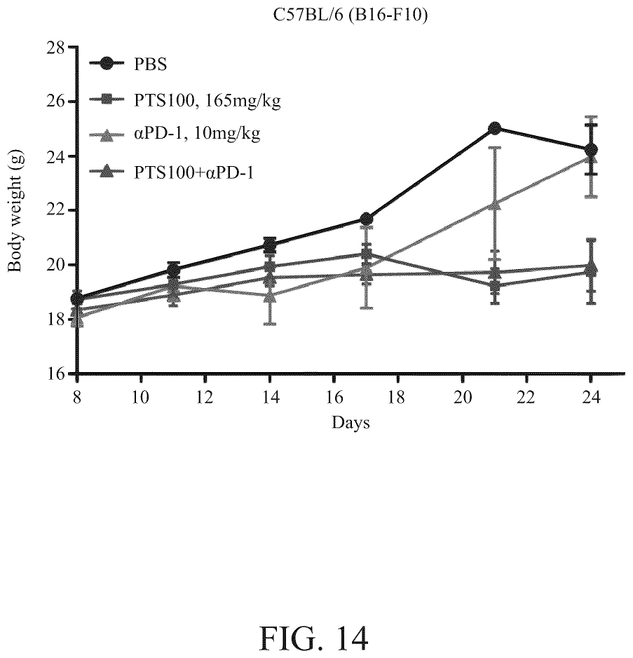

Drug administration started on Day 8. Since the high lethality of melanoma cell line B16-F10, mice began to die at 15 days after tumor inoculation. Therefore, although the mice were sacrificed on Day32, the final day of the body weight and tumor size being calculated was Day 24. On Day 24, the survival rates of mice in the PBS, PTS100 and αPD-1 groups were only 20%, 40% and 30%, respectively. However, the survival rate of mice in PTS100+αPD-1 group significantly increased to 70% (FIG. 13). Body weight of mice in PBS and αPD-1 groups significantly increased on Day24, which may be caused by the growth of tumor (i.e., the increased body weight came from the increase of tumor weight) (FIG. 14).

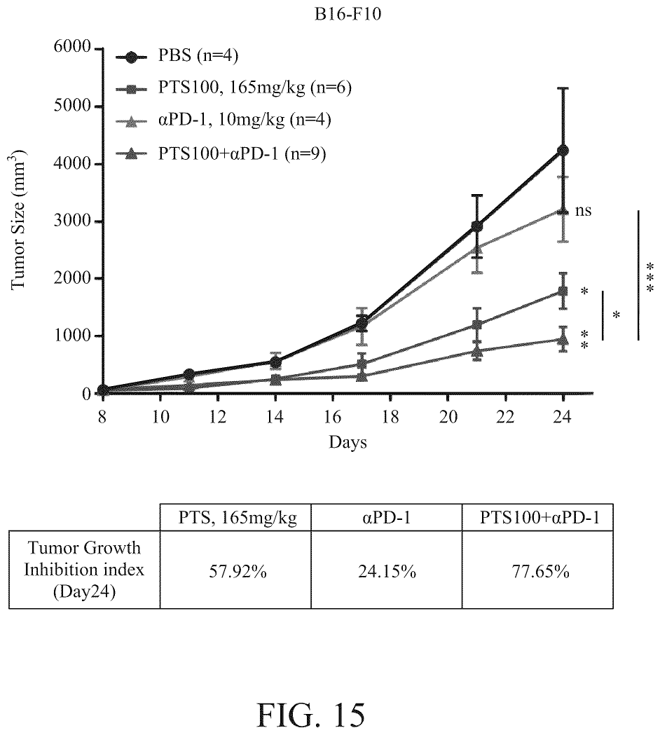

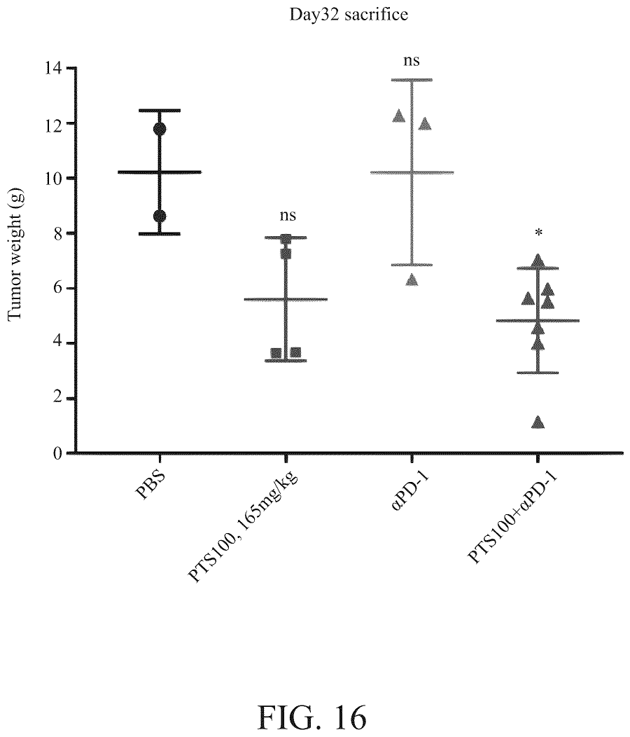

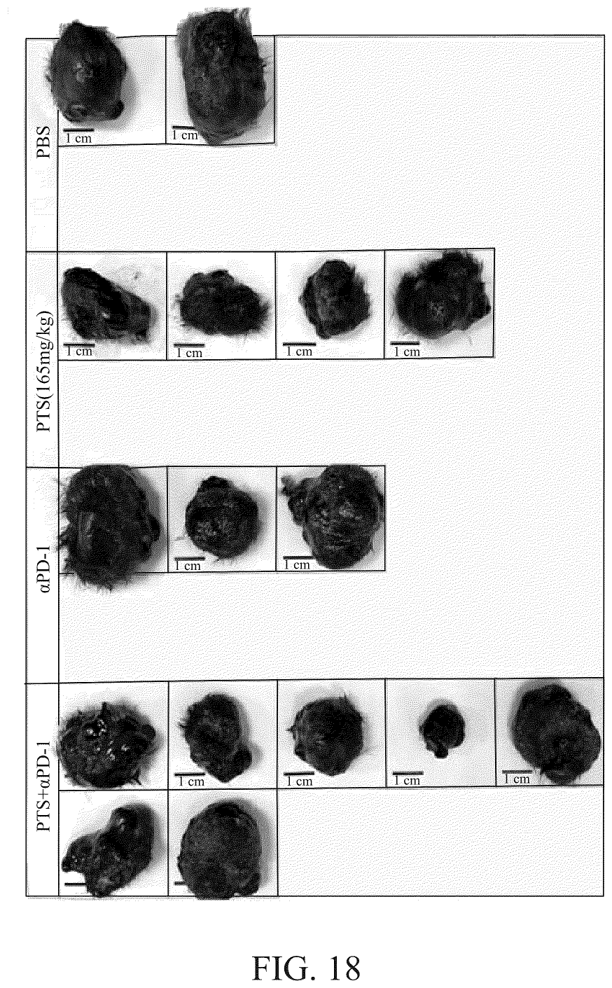

After the B16-F10 tumors were treated with PTS100, αPD-1, and PTS100+αPD-1, the tumor growth in the PTS100 and PTS100+αPD-1 groups were significantly reduced and the tumor growth inhibition indexes was 57.92% and 77.65%, respectively. While the tumor growth inhibition index of the αPD-1 group was only 24.15%, which suggested that using αPD-1 alone may not effectively inhibit the B16-F10 tumor (FIG. 15). Furthermore, the tumor weight in PTS100+αPD-1 group on Day 32 significantly reduced, but the inhibitions of αPD-1 alone and PTS100 alone on the tumor weight were not statistically significant may be due to the insufficient sample size on Day 32 (FIG. 16). FIG. 18 shows the tumor appearance in each treatment group of B16-F10 melanoma allograft mouse model.

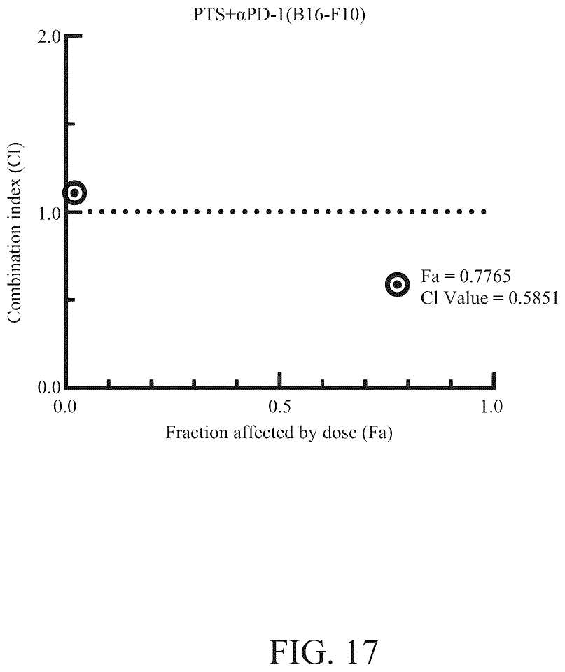

Through the calculation and analysis using the CompuSyn software, calculating the index of the synergistic effect of drug combination (Combination index, CI, CI=1, <1 and >1 indicates additive effect, synergism and antagonism, respectively), the CI value was 0.5851, confirming the synergistic effect of the drug (FIG. 17).

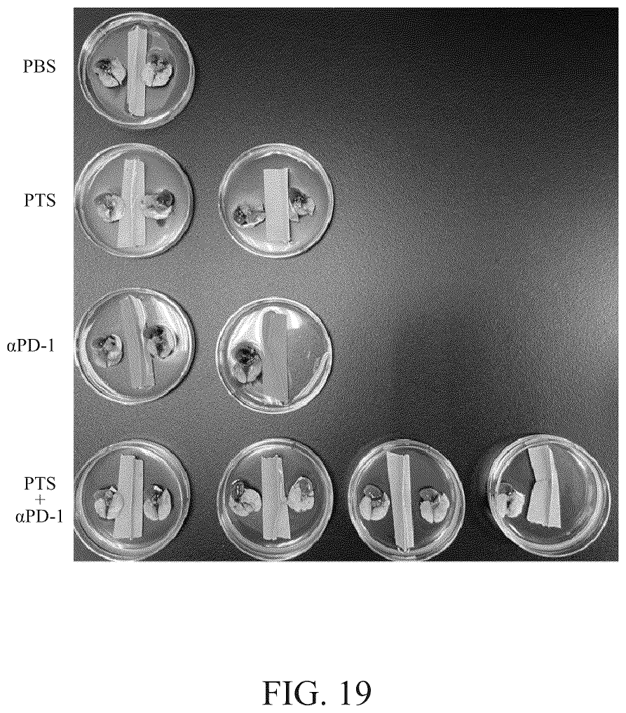

In all the groups, there was only one mouse in the PBS group being observed to has metastatic lung tumor on the day of sacrifice (FIG. 19), and thus the number of lung metastatic nodules was not calculated.

### CONCLUSION

No matter in the breast cancer or melanoma, the PTS100+αPD-1 combination treatment group showed significantly better tumor growth inhibition, tumor weight inhibition, and lung metastatic nodule number inhibition than PTS100 or αPD-1 treatment alone. Therefore, regardless of primary tumor or lung metastasis, the combined treatment of PTS100+αPD-1 obviously has a better therapeutic effect, and the increased survival rate in the melanoma allograft mouse model also indicates that tumors can be effectively inhibited by PTS100+αPD-1. The CompuSyn software algorithm also confirms that the combination has a synergistic effect. Furthermore, the number of lung metastatic nodules in the 4T1 breast cancer allograft mouse model was lower in the PTS100 group and the PTS100+αPD-1 group also indicates the anti-metastatic effect of PTS. In conclusion, the combination therapy of PTS with αPD-1 has a synergistic effect on the inhibition of localized cancers and cancer metastases, and thus can increase the overall survival of animals with cancers. The above-described descriptions of the detailed embodiments are to illustrate the preferred implementation according to the present disclosure, and it is not to limit the scope of the present disclosure. Accordingly, all modifications and variations completed by those with ordinary skill in the art should fall within the scope of the present disclosure defined by the appended claims.

What is claimed is:

1. A method of treating cancer, comprising administering a therapeutically effective amount of a pharmaceutical composition and an immunotherapeutic agent to a subject in need thereof, wherein the pharmaceutical composition comprises a benzenesulfonamide derivative and a pharmaceutically acceptable carrier thereof.

2. The method of claim 1, wherein the benzenesulfonamide derivative is represented by formula (I) below: 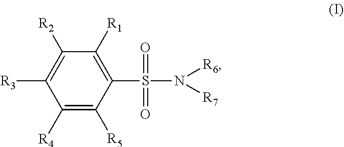
or a pharmaceutically acceptable salt thereof,
wherein R₁ to R₇ are independently selected from the group consisting of H, a C₁-C₆ linear or branched alkyl group, a C₁-C₆ linear or branched alkoxy group, a C₃-C₆ cycloalkyl group, a C₃-C₆ cycloheteroalkyl group, an amino group, and a halo group, or R₆ and R₇ are linked to each other to form a ring, and
wherein the alkyl group, the alkoxy group, the cycloalkyl group, the cycloheteroalkyl group, and the ring in R₁ to R₇ are independently unsubstituted or substituted with one or more substituents.

3. The method of claim 2, wherein the substituent is selected from the group consisting of phenyl, halo, oxo, ether, hydroxyl, carboxyl, amino, sulfo, and sulfonamide groups.

4. The method of claim 3, wherein the benezesulfonamide derivative or the pharmaceutically acceptable salt thereof is selected from the group consisting of para-toluene sulfonamide, ortho-toluene sulfonamide, meta-toluene sulfonamide, N-ethyl-ortho-toluene sulfonamide, N-ethyl-para-toluene sulfonamide, N-cyclohexyl-para-toluene sulfonamide, and any combination thereof.

5. The method of claim 1, wherein the benzenesulfonamide derivative is para-toluene sulfonamide.

6. The method of claim 1, wherein the immunotherapeutic agent is an immune checkpoint inhibitor (ICI).

7. The method of claim 6, wherein the ICI is selected from the group consisting of anti-PD-1 antibodies, anti-PD-L1 antibodies, anti-CTLA4 antibodies, anti-LAG-3 antibodies, anti-TIM-3 antibodies, and any combinations thereof.

8. The method of claim 1, wherein the cancer is a breast cancer or a melanoma.

9. The method of claim 1, wherein a ratio of the benzenesulfonamide derivative to the immunotherapeutic agent is in a range of 12.5:1 to 2000:1.

10. The method of claim 8, wherein a ratio of the benzenesulfonamide derivative to the immunotherapeutic agent is in a range of 1.5:1 to 100:1.

11. The method of claim 10, wherein the benzenesulfonamide derivative is administered to the subject at a dosage of about 55 mg/kg, and the immunotherapeutic agent is administered to the subject at a dosage of about 2 mg/kg.

12. The method of claim 10, wherein the benzenesulfonamide derivative is administered to the subject at a dosage of about 165 mg/kg, and the immunotherapeutic agent is administered to the subject at a dosage of about 2 mg/kg.

13. The method of claim 1, wherein the benzenesulfonamide derivative in the pharmaceutical composition is administered to the subject in an effective amount of from about 3,300 mg to about 26,400 mg.

14. The method of claim 1, wherein the benzenesulfonamide derivative in the pharmaceutical composition is administered to the subject in an effective amount of from about 165 mg to about 6,600 mg per day.

15. The method of claim 1, wherein the pharmaceutical composition is administered to the subject one time to five times a week.

16. The method of claim 1, wherein the pharmaceutically acceptable carrier is selected from the group consisting of a filler, a binder, a preservative, a disintegrating agent, a lubricant, a suspending agent, a wetting agent, a flavoring agent, a thickening agent, an acid, a biocompatible solvent, a surfactant, a complexation agent, and any combination thereof.

17. The method of claim 1, wherein the pharmaceutically acceptable carrier is selected from the group consisting of polyethylene glycol, alkylene glycol, propylene glycol, sebacic acid, dimethyl sulfoxide, ethanol, and any combination thereof.

18. The method of claim 1, wherein the pharmaceutical composition is in a form selected from the group consisting of a formulation to injection, dry powder, a tablet, an oral liquid, a wafer, a film, a lozenge, a capsule, a granule, a pill, a gel, a lotion, an ointment, an emulsifier, a paste, a cream, an eye drop, and a salve.

19. The method of claim 1, wherein the pharmaceutical composition or the immunotherapeutic agent is administered to the subject intratumorally, intravenously, subcutaneously, intradermally, orally, intrathecally, intraperitoneally, intranasally, intramuscularly, intrapleuraly, topically, or through nebulization.

20. The method of claim 1, wherein the subject is a human, a dog, a cat, or a mouse.
